(19) United States
(12) Patent Application Publication
(10) Pub. No.: US 2005/0059863 A1
(43) Pub. Date: Mar. 17, 2005

(54) Laryngoscope

(75) Inventor: Peter Zilch, Berlin (DE)

Correspondence Address: DENNISON, SCHULTZ, DOUGHERTY & MACDONALD, 1727 KING STREET, SUITE 105, ALEXANDRIA, VA, 22314, US

(73) Assignee: Schunk Metall- und Kunstoff GmbH

(21) Appl. No.: 10/927,534

(22) Filed: Aug. 27, 2004

(30) Foreign Application Priority Data
Sep. 1, 2003 (DE) 103 40 576.3
Sep. 9, 2003 (DE) 103 41 886.5

(51) Int. Cl. A61B 1/267

(52) U.S. Cl. 600/188; 600/199

(57) ABSTRACT

A laryngoscope including a handle and a spatula that is detachably connected thereto via a coupling. So that an image can be transmitted free of distortion over an image conductor from the spatula in the handle, spatula and handle are connected via first and second projections. The projections make it possible to twist the spatula in relation to the handle as well as snap-fit them to each other.

### BACKGROUND OF THE INVENTION

The invention relates to a laryngoscope, comprising a handle and a spatula that is detachably connected thereto via a coupling, a light-emitting element arranged in the spatula an image conductor running through the spatula, which in the area of the coupling can be aligned with a video input opening arranged in the handle, which is assigned to a video recording device arranged in the handle, wherein the coupling has at least one mechanical centering element for precisely aligning the image conductor with the video input opening.

An analogous laryngoscope can be found in DE 199 55 180 A1. In order to precisely align an image conductor extending from the spatula with a lens system arranged in the handle, the mechanical centering element has a cone as well as a corresponding countercone, of which one is arranged on the handle and the other on the spatula. These measures are intended to enable a rigid attachment of one to the other in a radial as well as an axial direction. Fit inaccuracies of a maximum of 0.1 mm. should also be guaranteed. Additionally further snap-fit devices are provided in the coupling area between spatula and handle, which preferably contain fastening nuts.

In a laryngoscope well known from EP 0 901 7472 B1, the spatula is connected to the handle by means of a bayonet clasp in order to enable image transmission from an image conductor running through the spatula to an image-recording unit arranged in the handle.

In practice it has been shown that the analogous types of coupling are not sufficient to adequately align the image conductor precisely with the video input opening existing in the handle in order to avoid image distortions and/or exclude that the picture drifts. With bayonet and screw couplings a quick change of spatulas is not feasible. A folded-up position, snug against the handle, for transport purposes is also not possible.

That is why in practice laryngoscopes have gained acceptance that, as a matter of principle, do not have any integrated image-recording units. EP 0 110 333 B1 or EP 0 030 014 B1, which contain lighting devices, can be referenced as examples.

A laryngoscope with a spatula that is connected to a handle is known from DE-B-1 766 713. A fiberglass light guide extends from the handle to inside the spatula, with a place for the light to exit in the distal area of the spatula. The spatula and handle are connected, for the purpose of centering the fiberglass light guide on the exit end of the light guide in the handle, by an axis with a flattened area and a borehole open at the side. The borehole is accessible from the outside by a slot. Furthermore, a catch spring is screwed onto the handle, which reaches beneath the rim of a neck for snap-fit purposes.

### SUMMARY OF THE INVENTION

The present invention is based on the object of designing a laryngoscope of the above kind in such a way that it ensures that the spatula and handle are aligned in relation to each other so precisely that image distortions are excluded, yet at the same time offer the possibility of detaching the tongue and handle from each other. Additionally, through simple design measures, the spatula shall be able to pivot on the handle without causing uncontrolled detaching. The precise alignment between handle and spatula can be implemented with simple measures, wherein it shall be possible to swivel and fix the spatula and handle with one hand.

In accordance with the invention, the problem is basically solved in that the mechanical centering element has at least one elongated first protrusion extending from the spatula or the handle, having a width B and a length L, to which a first recess provided in the handle or spatula is allocated, in that the first recess is composed of two sections, wherein a channel-type outer section that is suitable for the insertion of the first projection in the operating position of the spatula that is connected to the handle runs in a slanted fashion to the longitudinal axis of the handle and has a width that corresponds to the width B of the first projection, and an inner section that extends from the outer section has a circular geometry with a diameter that corresponds to the length L of the first projection, and in that furthermore the mechanical centering element has a least one snap-fit projection extending from the spatula or the handle, to which a second recess in the handle or the spatula is assigned in the operating position of the spatula that is attached to the handle, wherein said second recess can be snapped into the snap-fit projection.

According to the invention, it is guaranteed by simple design measures that the handle and spatula can be precisely aligned to one another so that the image conductor can be precisely aligned with the video input opening provided in the handle, by which precision levels in the order of magnitude of between one hundredth and two hundredths of a millimeter are possible. In this way it is guaranteed that the image received over the image conductor from the spatula can be transmitted image-recording device that is integrated in the handle.

The solution according to the invention offers the same handling as the traditional laryngoscopes. Thus it is not necessary for the user to readjust his handling. In addition, a folded-up position, snug against the handle, is possible for transport purposes, without the spatula and handle having to be detached from one another.

In further development of the invention it is provided that the spatula-side, thus the distal end of the handle is fork-shaped and has two legs between which a handle-side, thus proximal end-section, which can also be called a base, runs. As a result, the first projections can extend from the side of the proximal section of the spatula that is turned toward the socket, wherein the projections run, as far as their longitudinal axis is concerned, parallel to the longitudinal axis of the proximal end of the spatula. Moreover the snap-fit projections should likewise extend from the sides of the proximal end of the spatula facing the legs.

The first projections with the recesses assigned thereto and consisting of an inner and an outer section, and the snap-fit projections, which in turn likewise engage into corresponding recesses when the spatula is in operating position, form a coupling system, which enables mechanical centering without any need for cones or bayonet clasps. On the contrary, it can be guaranteed with simple design measures that the spatula is so precisely aligned with the handle that to the extent required the image, which is to be guided from the spatula to the image-recording device, is transmitted free of distortion, and without running the risk of the picture drifting. Through the precise alignment of spatula and handle it is also guaranteed that the required precise connection to the light-emitting element occurs in order to illuminate the area, which is to be optically recorded.

In further development it is provided that the light-emitting element is at least one light-emitting diode arranged in the spatula, in particular its distal area, which is connected via guide sections running in the spatula and the handle with a voltage source, which can be arranged in the handle if necessary. In the operating position of the spatula, the required electrical connection between the guide sections running in the spatula and the handle is then assured.

The snap-fit connection is in particular a spring-loaded spherical element, extending from the proximal section of the spatula, which runs between the legs of the distal end of the handle. As a result, each spring-loaded spherical element preferably penetrates one side of the proximal end of the spatula. For this purpose the spatula can have a borehole, which is caulked at the ends so the spherical elements cannot slip out. A spring element, such as a coil spring, is then situated between the spheres.

### BRIEF DESCRIPTION OF THE DRAWINGS

Further details, advantages, and characteristics of the invention are revealed not only from the claims, the characteristics revealed in them—either alone or in combination, but also from the present description of a preferred embodiment disclosed in the drawings, in which:

FIG. 1 is first sectional view of a laryngoscope;

FIG. 2 is a second sectional view of the laryngoscope according to FIG. 1;

FIG. 3 is a section of the handle and spatula of the laryngoscope according to FIGS. 1 and 2 shown spaced from each other;

FIG. 4 is a view corresponding to FIG. 3, in which the spatula is connected to the handle;

FIGS. 5 and 6 are views which elucidate the possibility of articulating the spatula in relation to the handle;

FIG. 7 is a top view onto the elements according to FIG. 3, partially broken away; and

FIG. 8 is an underneath view of the proximal end of the spatula of the laryngoscope according to FIGS. 1 and 2, partially broken away.

### DESCRIPTION OF THE PREFERRED EMBODIMENTS

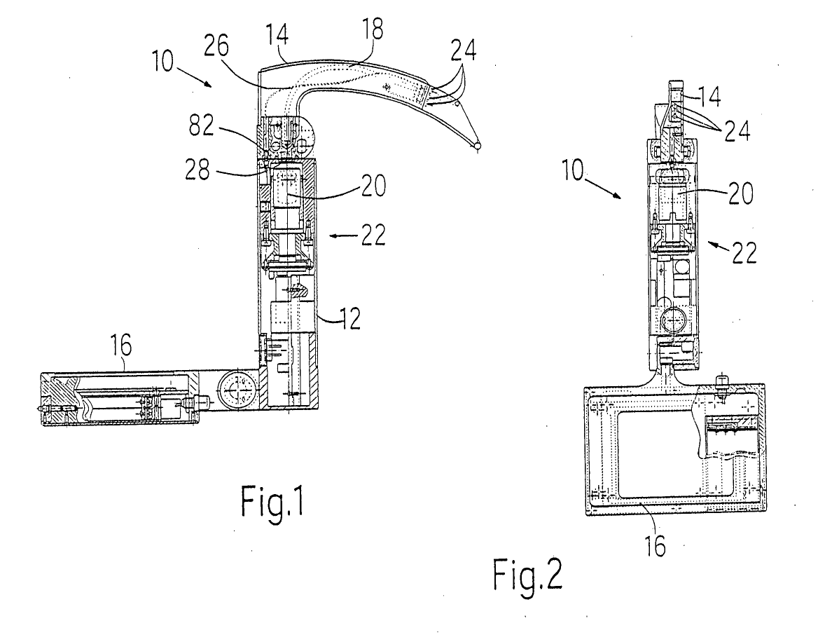

In FIGS. 1 and 2 the sectional views of a laryngoscope 10 are illustrated, which insofar represents a classic design that the laryngoscope 10 consists of a handle 12 and a spatula 14 that is detachably connectable thereto. An LCD monitor 16 extends detachably from the proximal end of the handle 12, wherein said monitor according to the illustrations in FIGS. 1 and 2 can pivot.

Images can be depicted on the screen of the LCD monitor 16, which are transmittable to an image processing unit 22 via an image conductor 18 and an optical element 20. As a result, a pictures are taken of the throat cavity of a patient by using the laryngoscope 10. For this purpose, the image conductor ends in the area of the distal end of the spatula 14. In addition, a light source is provided in the distal area of the spatula 14, in the form of a light-emitting diode 24, which is supplied with the required voltage via a guide 26. The voltage source can be located inside the handle 12.

To ensure that the image can be transmitted over the image conductor 18 free of distortion via an image input opening 28 to the optical element 20 without the risk of the image drifting, the spatula 14 and handle 12 must be so precisely aligned to one another that a precision of around 1/100 to 2/100 mm is attainable.

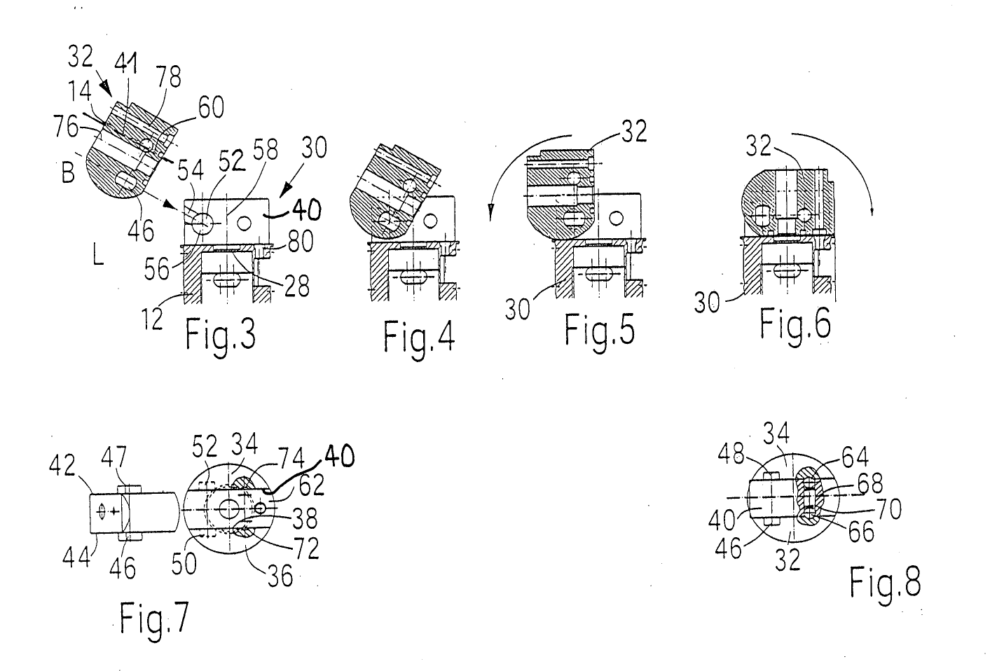

This is solved according to the invention by means of simple mechanical means, which guarantee the desired precision regardless. This should be elucidated by FIG. 3 to 8, in which the spatula-side or distal end 30 of the handle 12 and the handle-side or proximal end 32 of the spatula 14 are shown, which can also be called a base.

The distal end 30 of the handle 12 is fork-shaped and has two legs 34, 36 with inner surfaces 38, 40 running parallel to one another and facing one another. Between the legs 34, 36 the proximal end 32, or the socket of the spatula 14, can be arranged, which can have the shape of a rectangular parallelepiped.

In order to guarantee a precise alignment of handle 12 and spatula 14, i.e. of its distal end 30 or proximal end 32, and simultaneously guarantee a secure attachment, elongated projections 46, 48 extend from the proximal end 32, i.e. its outer surfaces 42, 44, which have a width B and a length L. The projections 46, 48 run parallel to the longitudinal axis 41 of the proximal section or base 32 of the spatula 14. The projections 46, 48 exhibit in a top view a rectangular shape with convexly running narrower legs.

First recesses 50, 52, the geometries of which are adapted to the projections 46, 48, run in the inner surfaces 38, 40 of the legs 34, 36 of the distal area 30 of the handle 12, each of which are composed of an outer section 54 and an inner section 56. The outer section 54 is channel-shaped and has a width that is equal to the width B of the projection 46, 48. The longitudinal direction of the outer channel-shaped section 52 runs in a slanted fashion to the longitudinal axis 58 of the handle 12. In particular, the longitudinal axis 58 of the handle 12 forms an angle of between 45 and 75 with the longitudinal axis of the outer channel-shaped section 54.

The outer section 54 transitions into the inner section 56, which has a circular shape with a diameter that corresponds to the length L of the projection 46, 48. Through design measures in this connection the spatula 14 can be inserted into the recesses 50, 52 in a position running at an angle to the longitudinal axis 58 of the handle 52 via the projections 46, 48, and then the spatula 14 can be pivoted about the axis specified by the center of the inner section 56 on the projections 46, 48, which are incorporated into the inner sections 56 of the recesses 50, 52, as is shown in FIG. 4, 5, 6.

In order to fix the spatula 14 on the handle 12 in the working position of the spatula 14, when its distal surface 60, and thus the front surface of the base 32, is flush with the surface 62 running between the legs 34, 36 of the handle 12, additionally snap-fit projections extend from the distal end 32 in the form of spring-loaded spherical elements 64, 66, which are arranged within a borehole 68 caulked on the outside and are supplied with a force by a spring element such as a coil spring 70 in the direction of the respective opening of the borehole 68. The spherical elements 64, 66 are assigned to corresponding second recesses 72, 74 in the inner surfaces 38, 40 of the legs 34, 36 in order to enable a snap-fit of the spatula 14 to the handle 12.

Naturally, the spherical elements 64, 66, with the recesses 72, 74 assigned to them could also be replaced by elements acting in the same way, e.g. by screws, which would then come under the terms of the invention.

The elongated projections 46, 48 with the recesses or niches 46, 48 assigned to each of them on an inner and an outer section 54, 56, as well as the snap-fit projections 46, 48 with the snap-fit recesses 72, 74 consequently form a coupling system, by which, in a simple yet secure manner, a precise alignment of spatula 14 to handle 12 is attained.

With the spatula 14 snapped into place, a borehole 76 receiving the light guide 18 is precisely aligned to the image input opening 28 running in the surface 62 of the handle 12 between the legs 34, 36 of the handle 12, so that as a consequence an image can be transmitted free of distortion over the image conductor 18 and the optical element 20 to the image recording unit 22. A prerequisite for this is, naturally, that the image conductor 18 is precisely arranged in the borehole 18. In particular, the image conductor 18, fitted into a sleeve, completely fills in the borehole 78 in order to preclude any relative movement. This guiding sleeve can also, by adjustment screws introduced into the base 32, be adjusted so that the image conductor 18 is exactly positioned.

The electric conductor 26 or sections thereof, as the case may be, run in truly aligned boreholes 78, 80 of the spatula 14 and the handle 12 when the spatula 14 is snap-fit. At the interface between the spatula 14 and the handle 12 an appropriate electrical coupling is provided, which in FIG. 1 z is marked basically with the reference number 82.

Through the design of the projections 46, 48 and the recesses 50, 52 assigned to them, on the one hand a precise alignment of the image conductor 18 with the image input opening 28 is guaranteed in the case of a snap-fit handle 14, and on the other hand, however, there is also the possibility of pivoting the spatula 14, corresponding to the view in FIG. 5, in such a way that the surfaces 60, 62 of spatula 14 and handle 12 are freely accessible to each other without running the risk that the spatula 14 can be removed uncontrolled from the handle 12. Therefore the spatula 14 can be transported without problems in a folded-up position snug against the handle 12.

The image conductor can be inserted into a pipe and is thus protected and fixed in the necessary shape and position.

Since the spatula 14 and the handle 12 must be cleaned with aseptic cleaners, these are enclosed in a watertight manner by appropriate seals, fittings, and sealing materials.

1. Laryngoscope (10), comprising a handle (12) and a spatula (14) detachably attached thereto via a coupling, a light-emitting element (24) arranged in the spatula, an image conductor (18) running in the spatula, which can be aligned in the area of the coupling with an image input opening (28) arranged in the handle, said opening being assigned to a image recording device (22) arranged in the handle, wherein the coupling has at leas one mechanical centering element (46, 48, 50, 52) for precise alignment of the image conductor with the image input opening, characterized in that the mechanical centering element has at least one elongated first projection (46, 48) extending from the spatula (14) or the handle (12) with a width B and a length L, which is assigned to a first recess (50, 52) existing in the handle or the spatula, in that the first recess is composed of two sections (54, 56), wherein a channel-shaped outer section (54), which is suitable for the insertion of the first projection, runs in an inclined fashion to the longitudinal axis (58) of the handle in the operating position of the spatula that is connected to the handle, and has a width that corresponds to the width B of the first projection, and an inner section (56) extending from the outer section (56) has a circular shape with a diameter that a corresponds to the length L of the first projection, and in that the mechanical centering element furthermore has at least one snap-fit projection (64, 66) extending from the spatula or the handle, to which a second recess (72, 74) is assigned in the handle or the spatula in the operating position of the spatula connected with the handle, with the snap-fit projection being able to be snapped into said second recess.

2. Laryngoscope according to claim 1, wherein the first recess (50, 52) and/or the second recess (72, 74) runs in the handle (12.).

3. Laryngoscope according to claim 1, wherein the spatula-side (distal) end (30) of the handle (12) is fork-shaped with two legs (34, 36), between which the handle-side (proximal) end (32) of the spatula (14) runs.

4. Laryngoscope according to claim 3, wherein the first and/or the second recess (50, 52, 72, 74) runs in insides (38) of the legs facing each other.

5. Laryngoscope according to claim 1, wherein the longitudinal axis of the first projection (46, 48) extends along the longitudinal axis of the handle-side section of the spatula (14) or the longitudinal axis (58) of the handle.

6. Laryngoscope according to claim 1, wherein the light-emitting element (24) is at least one light guide with a first strand running in the spatula (14) and a second strand running in the handle (12).

7. Laryngoscope according to claim 1, wherein the light-emitting element (24) is at least one light-emitting diode arranged in the spatula (14), especially in its distal area which is connected via guide sections running in the spatula and the handle (12) with a voltage source, which is 9 particularly arranged in the handle.

8. Laryngoscope according to claim 1, wherein the snap-fit projection (64, 66) is a spring-loaded spherical element, which is arranged displaceably in the handle-side section (32) of the spatula (14)

9. Laryngoscope according to claim 8, wherein a spherical element is arranged in each of the sides (42, 44) of the handle-side section (32) of the spatula (14) assigned to the legs (34, 36) of the handle (12).

10. Laryngoscope according to claim 1, wherein the image conductor (18) runs, at least in the distal area of the spatula (14), in a guiding sleeve, which in turn is arranged in a breakthrough, such as a borehole (78), ssigned to the image input opening (28) and can be aligned with the image input opening, e.g. via adjusting elements, such as screws.
